(19) United States
(12) Patent Application Publication
(10) Pub. No.: US 2016/0324547 A1
(43) Pub. Date: Nov. 10, 2016

(54) SPINAL CORRECTION SYSTEM AND METHOD

(71) Applicant: Warsaw Orthopedic, Inc., Warsaw, IN (US)

(72) Inventors: Keith E. Miller, Germantown, TN (US); William Alan Rezach, Atoka, TN (US)

(21) Appl. No.: 14/704,702

(22) Filed: May 5, 2015

(51) Int. Cl. A61B 17/70

(52) U.S. Cl. CPC A61B 17/7053; A61B 2017/00367

(57) ABSTRACT

A spinal construct comprises at least one tether including a first end connectable with a first fastener connected with spinal tissue and a second end connectable with a second fastener connected with spinal tissue. The at least one tether has a tension between the fasteners. A member defines an axis and includes an element disposed transverse relative to the axis. The element is engageable with the at least one tether. The member further includes a drive engageable with the dement to adjust the tension. Systems, implants and methods are disclosed.

### TECHNICAL FIELD

The present disclosure generally relates to medical devices for the treatment of musculoskeletal disorders, and more particularly to a surgical system for correction of a spine disorder.

### BACKGROUND

Spinal disorders such as scoliosis and other curvature abnormalities, kyphosis, degenerative disc disease, disc herniation, osteoporosis, spondylolisthesis, stenosis, tumor and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment of vertebrae may employ implants, such as, for example, spinal constructs. The spinal constructs, which may include tethers, rods and bone screws, engage vertebrae to position and align one or more vertebrae. This disclosure describes improvements over these prior technologies.

### SUMMARY

In one embodiment, a spinal construct is provided. The spinal construct comprises at least one tether including a first end connectable with a first fastener connected with spinal tissue and a second end connectable with a second fastener connected with spinal tissue. The at least one tether has a tension between the fasteners. A member defines an axis and includes an element disposed transverse relative to the axis. The element is engageable with the at least one tether. The member further includes a drive engageable with the element to adjust the tension. In some embodiments, systems, implants and methods are provided.

### BRIEF DESCRIPTION OF THE DRAWINGS

The present disclosure will become more readily apparent from the specific description accompanied by the following drawings, in which:

FIG. 1 is a side perspective view of components of one embodiment of a surgical system in accordance with the principles of the present disclosure;

FIG. 2 is a perspective view of components shown in FIG. 1;

FIG. 3 is a break away view of the components shown in FIG. 2;

FIG. 4 is a cross section view of the components shown in FIG. 3;

FIG. 5 is a cross section view of the components shown in FIG. 3;

FIG. 6 is a cutaway view of the components shown in FIG. 3;

FIG. 7 is a cutaway view of the components shown in FIG. 3;

FIG. 8 is a perspective view of components of one embodiment of a surgical system in accordance with the principles of the present disclosure disposed with vertebrae;

FIG. 9 is a perspective view of components of one embodiment of a surgical system in accordance with the principles of the present disclosure;

FIG. 10 is a perspective view of components of one embodiment of a surgical system in accordance with the principles of the present disclosure; and

FIG. 11 is a cutaway view of components of one embodiment of a surgical system in accordance with the principles of the present disclosure.

### DETAILED DESCRIPTION

The exemplary embodiments of the system and related methods of use disclosed are discussed in terms of medical devices for the treatment of musculoskeletal disorders and more particularly, in terms of a surgical system and method for correction of a spine disorder.

In some embodiments, the present surgical system includes components, such as, for example, a tether reel that can be employed with a method for deformity correction. In some embodiments, the present surgical system includes a device and method for adjusting tension on a tether between fixation points. In some embodiments, the present surgical system includes a device and method that allow a practitioner to adjust tension of a tether between fixation points for controlled correction of scoliosis. In some embodiments, the present surgical system includes a pedicle screw having a tensioning device. In some embodiments, the present surgical system includes a pedicle screw and a separate tensioning device located on a tether between vertebral fixation points. In some embodiments, the present surgical system includes a device and method that adjust tension by increasing and/or decreasing tension in a tether engaged with vertebral bodies. In some embodiments, the present surgical system is utilized with an anterior tether system.

In some embodiments, the present surgical system includes a device and method to adjust tension, which can include increasing or decreasing tension, in a tether placed between vertebral bodies and/or between two locations in a body, such as, for example, ribs or a pelvis.

In some embodiments, the present surgical system includes a horizontal tether reel for adjusting tension on a tether between vertebrae. In some embodiments, the horizontal tether reel comprises a portion of a spinal screw. In some embodiments, the horizontal tether reel comprises a portion of a spinal hook or other fixation device. In some embodiments, components of the present surgical system can be implanted anterior and/or anterior laterally in a vertebral body. In some embodiments, components of the present surgical system can be implanted posteriorly in vertebral pedicles, vertebral facets and/or a head of a rib.

In some embodiments, the tether reel comprises a bone screw having a reel including an eyelet and a drive including a lock. In some embodiments, the tether reel comprises a ring clip connecting the reel with the bone screw and/or a ring clip connecting the drive with the bone screw. In some embodiments, components of the tether reel can be connected to the bone screw via assembly pins, staking, swaging and/or welding of cover plates. In some embodiments, the tether reel comprises a bevel gear connecting the drive and the reel. In some embodiments, the tether reel comprises a worm gear connecting the drive and the reel. In some embodiments, the tether reel comprises a spring connected with the lock to bias the lock between an engaged position and a release position such that the drive can rotate relative to the bone screw. In some embodiments, the present surgical system includes a ratcheting mechanism to adjust tension in a tether.

In some embodiments, the present surgical system is employed with a method for adjusting a tether. In some embodiments, the method includes the step of actuating a lock to rotate a driver component. In some embodiments, the method includes the step of releasing the lock. In some embodiments, the method includes the step of engaging a bony anatomy with a bone screw. In some embodiments, the method includes the step of threading a tether through the eyelet. In some embodiments, the method includes the step of releasing the lock component to rotate the drive component to actuate a reel via a bevel gear. In some embodiments, the method includes the step of adjusting the tether by winding and unwinding the reel to increase and/or decrease a tension on the tether.

In some embodiments, the present surgical system includes a tensioning device having a tether reel configured as a separate component to a bone fixation device. In some embodiments, the tensioning device is disposed on a tether between bone fixation points to adjust a tension between the bone fixation points with the reel.

In some embodiments, one or all of the components of the present surgical system may be disposable, peekpack, pre-packed sterile devices. One or all of the components of the present surgical system may be reusable. The present surgical system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

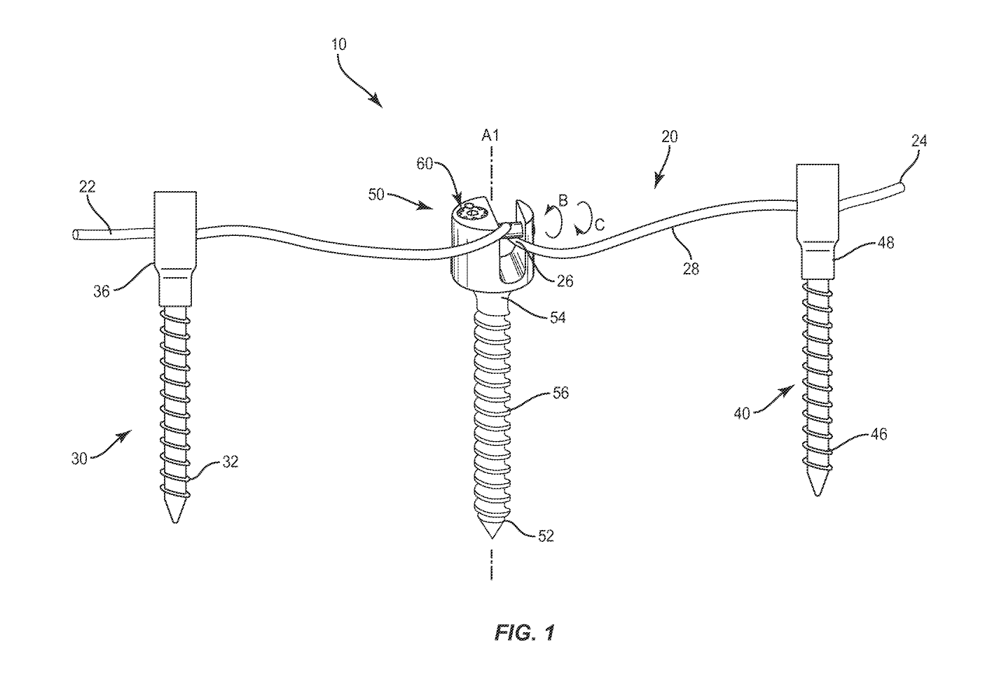

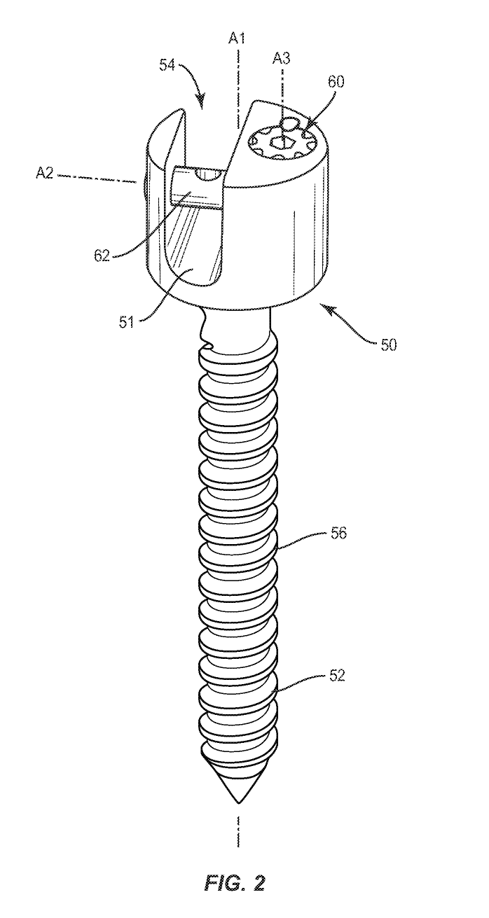

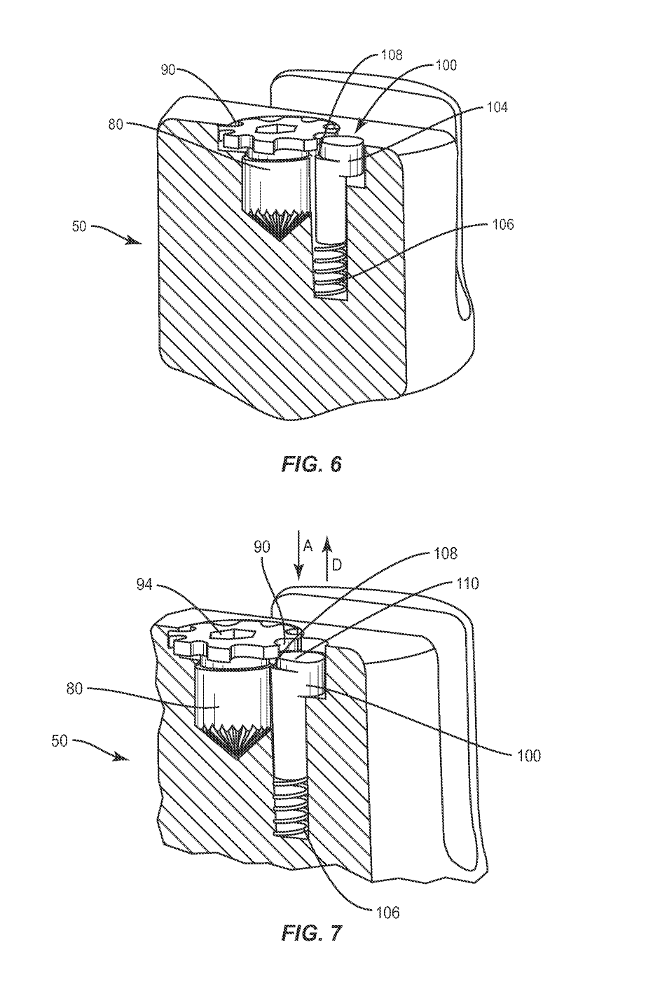

The following discussion includes a description of a surgical system in accordance with the principles of the present disclosure. Alternate embodiments are disclosed. Reference is made in detail to the exemplary embodiments of the present disclosure, which are illustrated in the accompanying figures. Turning to FIGS. 1-7, there are illustrated components of a surgical system, such as, for example, a spinal correction system 10.

The components of spinal correction system 10 can be fabricated from biologically acceptable materials suitable for medical applications, including metals, synthetic polymers, ceramics, bone material, tissue and/or their composites. For example, the components of spinal correction system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TOP), hydroxyapatite (HA)-TCP, calcium sulfate, or other resorbable polymers such as polyaetide, polyglycolide, polytyrosine carbonate, polycaroplaetohe and their combinations.

Various components of spinal correction system 10 may have material composites, including the above materials, to achieve various desired characteristics such as strength, rigidity, elasticity, compliance, biomechanical performance, durability and radiolucency or imaging preference. The components of spinal correction system 10, individually or collectively, may also be fabricated from a heterogeneous material such as a combination of two or more of the above-described materials. The components of spinal correction system 10 may be monolithically formed, integrally connected or include fastening elements and/or instruments, as described herein.

Spinal correction system 10 includes a longitudinal element, such as, for example, a tether 20 extending between an end 22 and an end 24. Tether 20 has a tension or tensile force measured between ends 22. 24. In some embodiments, the tension of tether 20 between ends 22, 24 is defined as a tensile force, for example, measured in Newtons (N) or pounds, which is a puffing force exerted on tether 20 on ends 22, 24 to draw tether 20 taut and to apply the tension in the direction of tether 20 to selected vertebrae for treating a spine, as described herein. Ends 22, 24 are connected to the selected vertebrae via fasteners, as described herein, such that the selected vertebrae and the spine experience a pulling force equal to the tension.

Tether 20 includes an intermediate portion 26 disposed between ends 22, 24. Tether 20 has a flexible configuration, which includes movement in a lateral or side to side direction and prevents expanding and/or extension in an axial direction upon fixation with vertebrae, as described herein. In some embodiments, all or only a portion of tether 20 may have a semi-rigid, rigid or elastic configuration, and/or have elastic properties, such as the elastic properties corresponding to the material examples described above, such that tether 20 provides a selective amount of expansion and/or extension in an axial direction. In some embodiments, tether 20 may be compressible in an axial direction. Tether 20 can include a plurality of separately attachable or connectable portions or sections, such as bands or loops, or may be monolithically formed as a single continuous element.

Tether 20 has an outer surface 28 and a uniform thickness/diameter. In some embodiments, outer surface 28 may have various surface configurations, such as, for example, rough, threaded for connection with surgical instruments, arcuate, undulating, porous, semi-porous, dimpled, polished and/or textured. In some embodiments, the thickness defined by tether 20 may be uniformly increasing or decreasing, or have alternate diameter dimensions along its length. In some embodiments, tether 20 may have various cross section configurations, such as, for example, oval, oblong, triangular, rectangular, square, polygonal, irregular, uniform, non-uniform, variable and/or tapered.

In some embodiments, tether 20 may have various lengths. In some embodiments, tether 20 may be braided, such as a rope, or include a plurality of elongated elements to provide a predetermined force resistance. In some embodiments, tether 20 may be made from autograft and/or allograft, as described herein, and be configured for resorbable or degradable applications.

In some embodiments, the longitudinal element may include one or a plurality of flexible wires, staples, cables, ribbons, artificial and/or synthetic strands, rods, plates, springs, and combinations thereof. In one embodiment, the longitudinal element is a cadaver tendon. In one embodiment, the longitudinal element is a solid core. In one embodiment, the longitudinal element is tubular.

Spinal correction system 10 includes a fastener, such as, for example, a bone screw 30 connected with spinal tissue and connectable with end 22 of tether 20. Spinal correction system 10 includes a fastener, such as, for example, a bone screw 40 connected with spinal tissue and connectable with end 24 of tether 20. Tether 20 has a tension between bone screws 30, 40, as described herein. Bone screws 30, 40 may be monolithically formed, integrally connected or attached with fastening elements to tether 20. In some embodiments, one or a plurality of bone screws may be connected with tether 20.

Bone screws 30, 40, are configured for penetrating fixation with tissue of vertebrae along a plurality of vertebral levels. Each of bone screws 30, 40, is disposed to engage a separate vertebral level. In some embodiments, one or a plurality of fasteners may be employed with a single vertebral level. In some embodiments, bone screws 30, 40 may be engaged with vertebrae in various orientations, such as, for example, series, parallel, offset, staggered and/or alternate vertebral levels. In some embodiments, the fasteners may include one or a plurality of anchors, tissue penetrating screws, conventional screws, expanding screws, wedges, anchors, buttons, hooks, clips, snaps, friction fittings, compressive fittings, expanding rivets, staples, nails, adhesives, posts, fixation plates and/or posts. These fasteners may be coated with an osteoinductive or osteoconductive material to enhance fixation, and/or include one or a plurality of therapeutic agents.

In some embodiments, spinal correction system 10 includes a member, such as, for example, a receiver 50. Receiver 50 is connected with a fastener, such as, for example, a bone screw 52 having a threaded shaft 56 configured for penetrating engagement with spinal tissue, such as, for example, vertebrae between bone screw 30 and bone screw 40. In some embodiments, receiver 50 may be monolithically formed, integrally connected or attached with fastening elements to bone screw 52. Receiver 50 defines a longitudinal axis A1. Receiver 50 includes a surface 51 that defines a cavity 54. Cavity 54 is configured for disposal of tether 20.

Receiver 50 includes a tether reel 60 that is mounted therewith. The components of tether reel 60 are engageable with a portion of tether 20, such as, for example, intermediate portion 26 to adjust the tension of tether 20, as described herein. In some embodiments, tether reel 60 is configured to selectively adjust the tension of tether 20. In some embodiments, tether reel 60 comprises a ratchet engageable with a drive 80, as described herein, to selectively adjust the tension, which includes increasing tension in a first direction and resisting and/or preventing decreasing tension in a second direction. In some embodiments, receiver 50, including tether reel 60, is connected with bone screw 30, which is connected with end 22 and/or bone screw 40, which is connected with end 24, similar to bone screw 52 as described herein, to adjust the tension of tether 20, as described herein. In some embodiments, a receiver 50, including tether reel 60, is connected with bone screw 30, a receiver 50, including tether reel 60, is connected with bone screw 40 and bone screw 52 does not include receiver 50, including tether reel 60. In some embodiments, a receiver 50, including tether reel 60, is connected with bone screw 30, or a receiver 50, including tether reel 60, is connected with bone screw 40 and bone screw 52 does not include receiver 50, including tether reel 60.

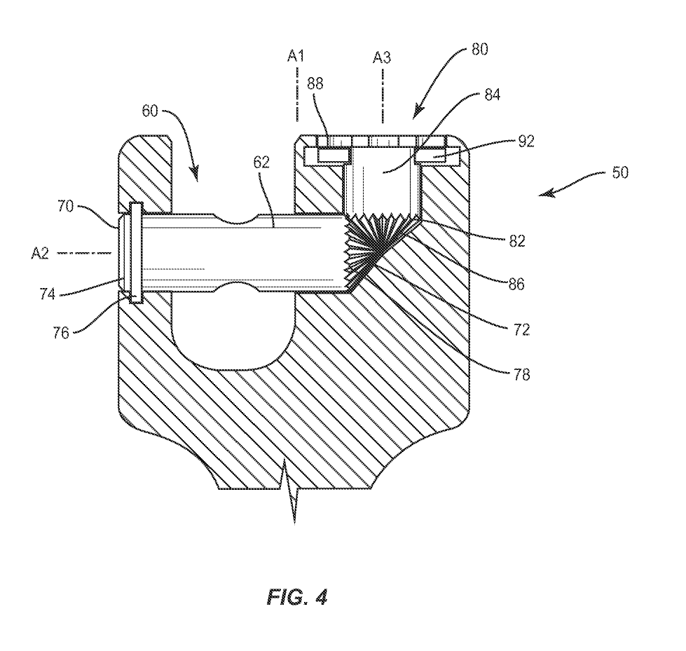

Tether reel 60 includes an element, such as, for example, a reel 62 engageable with tether 20 to selectively adjust the tension of tether 20. Reel 62 includes a cylindrical configuration and an outer surface 68. Re& 62 defines an axis A2 that is disposed transverse to axis A1, as shown in FIG. 4. In some embodiments, axis A2 is disposed perpendicular relative to axis A1. In some embodiments, this configuration provides a controlled tensioning of tether 20 and facilitates positioning of receiver 50 in a plurality of locations with vertebrae, such as, for example, anteriorly, anterior laterally and/or posteriorly. In some embodiments, axis A2 is disposed at alternate orientations, relative to axis A1, such as, for example, angular orientations such as acute or obtuse, and/or may be offset.

Reel 62 extends between an end 70 and an end 72. End 70 is disposed with an opening 74 of receiver 50. In some embodiments, receiver 50 includes a bearing, such as, for example, a ring clip 76 that supports red 62 with receiver 50. Reel 62 rotates within clip 76 and facilitates rotation of reel 62 relative to receiver 50 to selectively adjust the tension of tether 20.

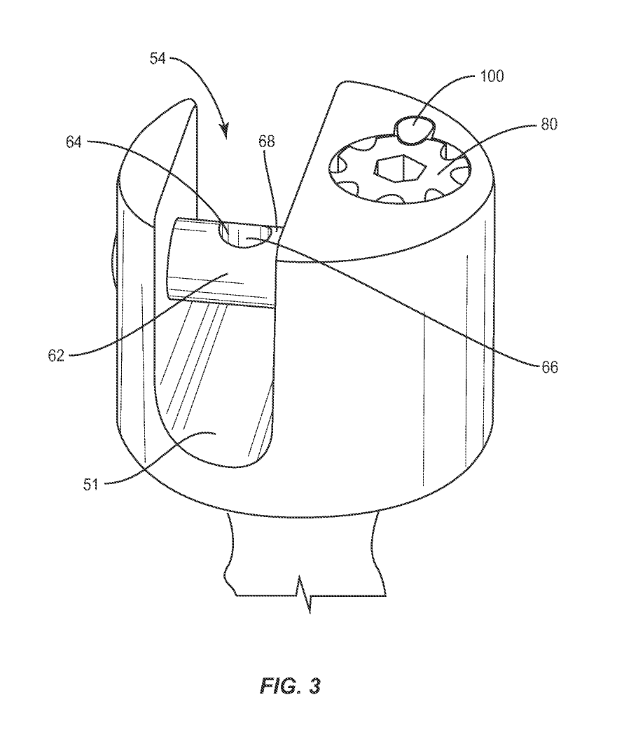

Reel 62 includes an inner surface 64 defining a cavity, such as, for example, an eyelet 66, as shown in FIG. 3, configured for disposal of tether 20. Tether 20, for example, intermediate portion 26, passes through eyelet 66. Rotation of reel 62 relative to receiver 50 disposes, encircles and/or winds intermediate portion 26 about surface 68 such that tether 20 is wound and/or unwound with reel 62. Rotation of reel 62 adjusts the tension of tether 20 between ends 22, 24.

End 72 includes a gear surface configured to engage a gear surface of drive 80, as described herein, to actuate rotation of reel 62 and adjust the tension of tether 20. In some embodiments, end 72 includes a gear surface, such as, for example, a bevel gear 78 of a bevel gear configuration, as shown in FIG. 4. In some embodiments, bevel gear 78 engages a bevel gear 86 of drive 80, as described herein, to actuate rotation of reel 62 and adjust the tension of tether 20.

In some embodiments, reel 62 is alternately configured, such as, for example, oval, oblong, rectangular, triangular, square, polygonal, irregular, uniform, non-uniform, offset, staggered, undulating, arcuate, variable and/or tapered. In some embodiments, outer surface 68 may include one or a plurality of openings. In some embodiments, all or only a portion of outer surface 68 may have alternate surface configurations, such as, for example, smooth, rough, arcuate, undulating, mesh, porous, semi-porous, dimpled and/or textured.

Drive 80 extends between an end 82 and an end 84. Drive 80 defines an axis A3 that is disposed transverse to axis A2, as shown in FIG. 4. In some embodiments, axis A3 is disposed perpendicular relative to axis A2, and axis A3 is disposed parallel and offset to axis A1. In some embodiments, this configuration provides a controlled tensioning of tether 20 and facilitates positioning of receiver 50 in a plurality of locations with vertebrae, such as, for example, anteriorly, anterior laterally and/or posteriorly. In some embodiments, axis A3 is disposed at alternate orientations, relative to axis A1 and/or axis A2, such as, for example, angular orientations such as acute or obtuse, and/or may be offset.

End 84 is disposed with an opening 88 of receiver 50, as shown in FIGS. 4-8. In some embodiments, receiver 50 includes a bearing, such as, for example, a ring clip 92 that supports drive 80 with receiver 50. Drive 80 rotates within ring clip 92 and facilitates rotation of drive 80 relative to receiver 50 to selectively adjust the tension of tether 20. In some embodiments, the components of tether reel 60 can be assembled with receiver 50 via other mechanisms, such as, for example, assembly pins, staking, swaging and/or welding.

End 82 includes bevel gear 86 that engages bevel gear 78 of the bevel gear configuration of tether reel 60 to actuate rotation of reel 62 and adjust the tension of tether 20. In some embodiments, the pitch surface of gears 78, 86 are conical, and reel 62 and drive 80 are disposed such that axes A2, A3 intersect. In some embodiments, the pitch surface of gears 78, 86 and axes A2, A3, respectively, each define a pitch angle of less than 90 degrees such that gears 78, 86 comprise cone-shaped external gears. In some embodiments, the pitch surface of gears 78, 86 and axes A2, A3, respectively, each define a pitch angle of greater than ninety degrees such that gears 78, 86 comprise internal bevel gears. In some embodiments, the pitch surface of gears 78, 86 and axes A2, A3, respectively, each define a pitch angle of exactly 90 degrees such that gears 78, 86 comprise crown gears. In some embodiments, the bevel gear configuration of tether reel 60 may comprise miter gears, skew bevel gears, straight bevel gears, spiral bevel gears, zero bevel gears and/or hypoid bevel gears.

End 84 includes a gear surface 90 that is configured for engagement with a lock 100 to resist and/or prevent movement of drive 80, as described herein. End 84 includes a cavity, such as, for example, a hexagonal socket 94 configured for a mating engagement with a surgical instrument, such as, for example, a driving tool (not shown) to rotate drive 80 and reel 62, connected via a gear surface configuration as described herein, relative to receiver 50 to adjust the tension of tether 20. In some embodiments, socket 94 may be variously configured, such as, for example, Phillips head, slotted head, hex socket head, hexagon external head, frearson head, square socket, square slotted combination head, spanner drilled tamper proof head and combinations thereof.

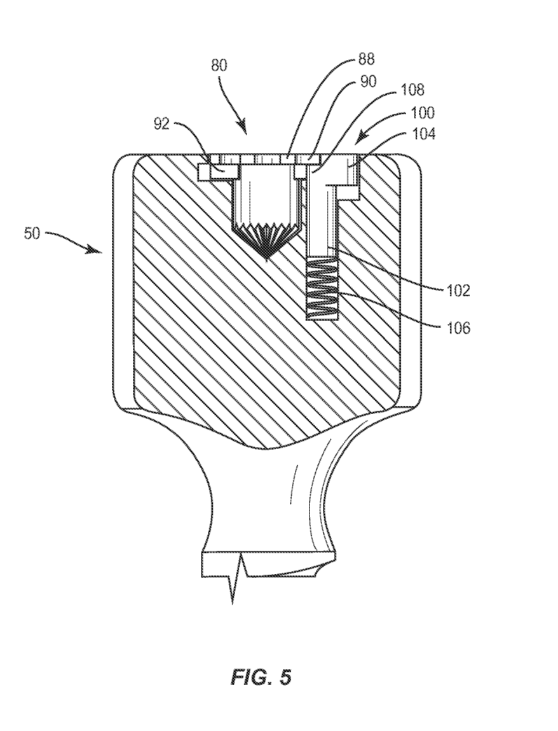

Lock 100 extends between an end 102 and an end 104. End 102 is configured for engagement with a resilient member, such as, for example, a spring 106 to bias lock into a first, locked configuration, as shown in FIG. 5 and described herein. End 104 includes a flange, such as, for example, a pawl 108 configured to engage a cylindrical gear surface 90 of drive 80 such that drive 80 is disposed in the locked configuration. With drive 80 disposed in the locked configuration, reel 62, connected to drive 80 via a gear surface configuration as described herein, is locked in position such that rotation thereof is resisted and/or prevented, as described herein.

End 104 includes a surface 110 configured for engagement with a surgical instrument and/or manipulation to move lock 100 between the locked configuration, such that movement of reel 62 is prevented and a second, non-locked configuration, as shown in FIG. 7, such that drive 80 and reel 62 are rotatable relative to receiver 50 to adjust the tension of tether 20. A force is applied to surface 110 to overcome a resistance of spring 106 to translate lock 100 between the locked configuration and the non-locked configuration.

In operation, tether 20 is inserted into cavity 54 and passed through eyelet 66, as shown in FIG. 1. A force is applied to surface 110, in a direction shown by arrow A in FIG. 7, to overcome the resilient bias of spring 106 and dispose lock 100 in the non-locked configuration. In some embodiments, a driving tool (not shown) may apply the force to surface 110 and engage socket 94 simultaneously for rotating drive 80. In some embodiments, a driving tool (not shown) may apply the force to surface 110 and engage socket 94 subsequently for rotating drive 80.

In the non-locked configuration, pawl 108 translates out of engagement with gear surface 90 such that the driving tool rotates drive 80. Drive 80 rotates reel 62 to adjust the tension of tether 20. With lock 100 disposed in the non-locked configuration, reel 62 is rotated, in a direction shown by arrow B and/or in a direction shown by arrow C in FIG. 1, to adjust tension of tether 20, which can include increasing and/or decreasing tension between bone screw 30 and bone screw 40. In some embodiments, reel 62 is rotated in the direction shown by arrow B to increase tension in tether 20 and reel 62 is rotated in the direction shown by arrow C to decrease tension in tether 20. The force applied to surface 110 is released such that lock 100 translates, in a direction shown by arrow D in FIG. 7, and the resilient bias of spring 106 disposes lock 100 in the locked configuration to resist and/or prevent movement of drive 80 and reel 62.

In assembly, operation and use, spinal correction system 10, similar to the systems and methods described herein, is employed with a surgical procedure, such as, for example, a correction treatment to treat adolescent idiopathic scoliosis and/or Scheuermann's kyphosis of a spine. In some embodiments, one or all of the components of spinal correction system 10 can be delivered or implanted as a pre-assembled device or can be assembled in situ. Spinal correction system 10 may be completely or partially revised, removed or replaced. In some embodiments, spinal correction system 10 includes fusionless treatment of vertebrae.

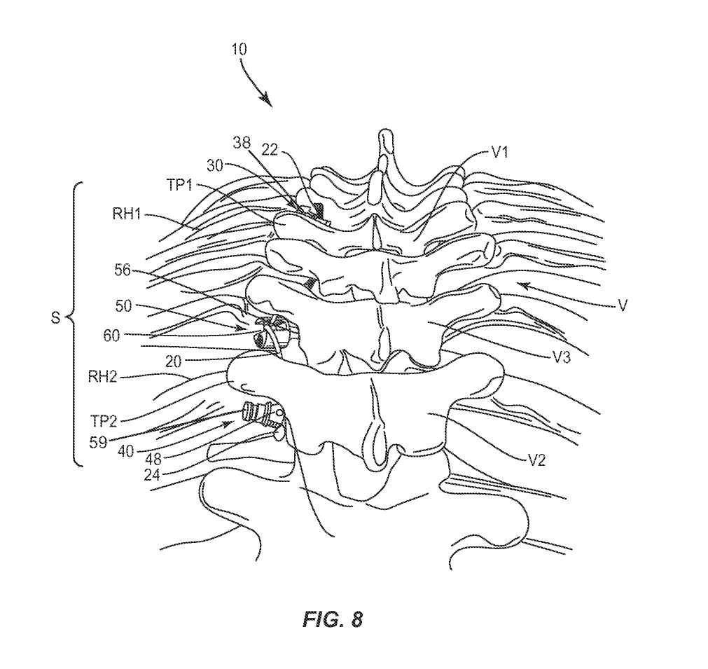

For example, as shown in FIG. 8, spinal correction system 10 can be employed with a surgical correction treatment of an applicable condition or injury of an affected section of a spinal column and adjacent areas within a body, such as, for example, a vertebra V1, a vertebra V2 and a plurality of vertebra V3 disposed therebetween.

In use, to treat a selected S of vertebrae V, a medical practitioner obtains access to a surgical site including vertebrae V in any appropriate manner, such as through incision and retraction of tissues. In some embodiments, spinal correction system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder. In some embodiments, the configuration and dimension of tether 20 is determined according to the configuration and dimension of selected section S.

An incision is made in the body of a patient and a cutting instrument (not shown) creates a surgical pathway along a selected approach, for example, as described herein, for implantation of components of spinal correction system 10 within the patient body. In some embodiments, a posterior surgical approach is employed and a sleeve or cannula is used to access a costo-vertebral space adjacent vertebra V1 to facilitate delivery and access for components of spinal correction system 10 along the surgical pathway, for example, such that tether 20 is disposed in a costo-vertebral space between costo-vertebral surfaces, which include surfaces of a transverse process TP1, a rib head RH1 and vertebra V1. In some embodiments, an anterior approach or an anterior lateral approach can be employed for access to an anterior surface of vertebrae V. A preparation instrument (not shown) can be employed to prepare tissue surfaces of vertebrae V and adjacent rib surfaces, as well as for aspiration and irrigation of the region.

Bone screw 30 is configured to support a tensile load with tether 20 over costo-vertebral surfaces, which include surfaces of transverse process TP1 and rib head RH1. A pilot hole is made in vertebra V1 for receiving bone screw 30. Bone screw 30 is delivered along the surgical pathway adjacent the costo-vertebral space for penetrating engagement with vertebra Vi. Bone screw 30 includes a threaded bone engaging portion 32 that is inserted or otherwise engaged with vertebra V1. Bone screw 30 has a head 36 with a bore, or through opening and a set screw 38, which is torqued onto tether 20 such that end 22 of tether 20 is connected with bone screw 30.

A pilot hole is made in vertebra V3 for receiving bone screw 50. Bone screw 50 is delivered along the surgical pathway, or an alternate surgical pathway along a substantially posterior approach, adjacent a costo-vertebral space for penetrating engagement with vertebra V3. Shaft 56 of bone screw 50 is inserted or otherwise engaged with vertebra V3.

Bone screw 40 is configured to support a tensile load with tether 20 over costo-vertebral surfaces, which include surfaces of a transverse process TP2, a rib head RH2 and vertebra V2. A pilot hole is made in vertebra V2 for receiving bone screw 40. Bone screw 40 is delivered along the surgical pathway, or an alternate surgical pathway along a substantially posterior approach, adjacent a costo-vertebral space for penetrating engagement with vertebra V2. Bone screw 40 includes a threaded bone engaging portion 46 that is inserted or otherwise engaged with vertebra V2. Bone screw 40 has a head 48 with a bore, or through opening and a set screw 59, which is torqued on to tether 20.

Tether 20 is delivered along the surgical pathway to the costo-vertebral space for attachment with bone screw 30. Set screw 38 of head 36 is torqued on to the portion of tether 20 disposed with bone screw 30 to securely fix end 22 of tether 20 with vertebra V1. Tether 20 is disposed in the costo-vertebral space on a posterior side of vertebra V1 such that tether 20 is disposed on an anterior side of transverse process TP1.

Tether 20 is threaded in a costo-transverse orientation along vertebra V1 and one or a plurality of vertebra V3 to dispose intermediate portion 26 of tether 20 in eyelet 66 of reel 62. Tether 20 is disposed in the costo-vertebral space on a posterior side of vertebra V3 such that tether 20 is disposed on an anterior side of transverse process TP3.

Tether 20 is threaded in a costo-transverse orientation along vertebra V3 and vertebra V2. Set screw 59 of head 48 is torqued on to the portion of tether 20 disposed with bone screw 40 to securely fix end 24 of tether 20 with vertebra V2. Tether 20 is disposed in the costo-vertebral space on a posterior side of vertebra V2 such that tether 20 is disposed on an anterior side of transverse process TP2.

Tether 20 is inserted into cavity 54 and passed through eyelet 66, as shown in FIG. 1. To tension tether 20 with tether reel 60, force is applied to lock 100 to dispose lock 100 in the non-locked configuration, as shown and described with regard to FIG. 7. In the non-locked configuration, a driving tool engages and rotates drive 80, as described herein. Drive 80 rotates reel 62 to adjust the tension of tether 20.

With lock 100 disposed in the non-locked configuration, reel 62 is rotated, in the direction shown by arrow B in FIG. 1, to increase tension in tether 20 and reel 62 is rotated, in the direction shown by arrow C in FIG. 1, to decrease tension in tether 20. Tether 20 is adjusted to correct vertebrae V at a selected tension between bone screws 30, 40. Lock 100 is disposed in the locked configuration, as described herein, to resist and/or prevent movement of drive 80 and reel 62.

The components of spinal correction system 10 are attached with a first side, such as, for example, a convex side of vertebrae V to prevent growth of selected section S, while allowing for growth and adjustments to a second side, such as, for example, a concave side of vertebrae V to provide treatment. Compression of section S of vertebrae V occurs along the convex side. In some embodiments, spinal correction system 10 implants components along a sagittal plane of a patient such that tether 20 is disposed anterior to a pedicle to reduce undesired lordosis. In some embodiments, spinal correction system 10 implants components along a coronal plane of a patient whereby tether 20 is disposed in a lateral orientation relative to a pedicle to provide correction in the coronal plane.

In one embodiment, spinal correction system 10 includes an agent, which may be disposed, packed, coated or layered within, on or about the components and/or surfaces of spinal correction system 10. In some embodiments, the agent may include bone growth promoting material, such as, for example, bone graft In enhance fixation of the fixation elements with vertebrae V.

Upon completion of the procedure, the surgical instruments, assemblies and non-implanted components of spinal correction system 10 are removed and the incision is closed. Spinal correction system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal correction system 10. In some embodiments, spinal correction system 10 may include one or a plurality of plates, connectors and/or bone fasteners for use with a single vertebral level or a plurality of vertebral levels.

In some embodiments, the components of spinal correction system 10 and methods of use as described herein for tethering of deformities may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal correction system 10 and methods of use as described herein may be used to prevent or minimize curve progression in individuals of various ages.

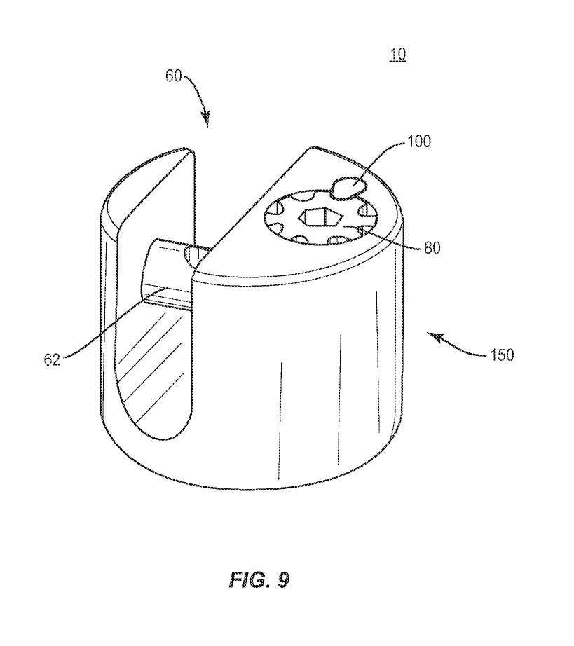

In one embodiment, as shown in FIG. 9, spinal correction system 10, similar to the systems and methods described with regard to FIGS. 1-7, includes a receiver 150, similar to receiver 50 described herein. Receiver 150 comprises the components of tether reel 60, which includes reel 62, drive 80 and lock 100 to selectively adjust the tension of tether 20, as described herein. Receiver 150 is free floating and not fixed with vertebrae. Receiver 150 is disposed between bone screws 30, 40, and in engagement with intermediate portion 26 of tether 20 to tension tether 20 therebetween.

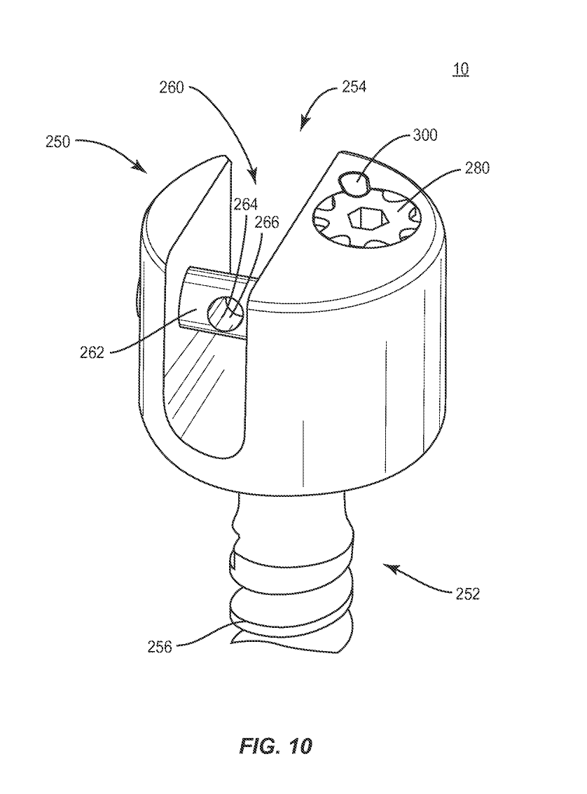

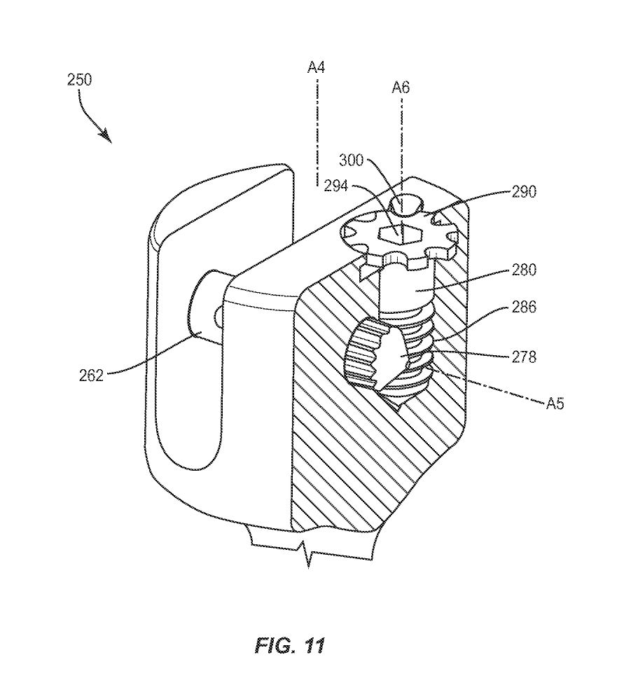

In one embodiment, as shown in FIGS. 10 and 11, spinal correction system 10, similar to the systems and methods described with regard to FIGS. 1-7, includes a receiver 250, similar to receiver 50 described herein. Receiver 250 is connected with a bone screw 252 having a threaded shaft 256 configured for penetrating engagement with vertebrae between bone screw 30 and bone screw 40. Receiver 250 defines a longitudinal axis A4. Receiver 250 defines a cavity 254 for disposal of tether 20.

Receiver 250 includes a tether reel 260, similar to tether reel 60 described herein, engageable with intermediate portion 26 to adjust the tension of tether 20, similar to that described herein. Tether reel 260 includes a reel 262, similar to reel 62 described herein, engageable with tether 20. Reel 262 defines an axis A5 that is disposed perpendicular relative to axis A4. Reel 262 includes an inner surface 264 defining an eyelet 266 configured for disposal of tether 20. Rotation of reel 262 relative to receiver 250 disposes, encircles and/or winds intermediate portion 26 about reel 262 such that tether 20 is wound and/or unwound with reel 262. Rotation of reel 262 adjusts the tension of tether 20 between ends 22, 24.

A drive 280 defines an axis A6 that is disposed perpendicular relative to axis A5, and axis A6 is disposed parallel and offset to axis A4. Tether reel 260 comprises a worm gear configuration, which includes a worm wheel gear 278 of reel 262 that engages a screw gear 286 of drive 280 to actuate rotation of reel 262 and adjust the tension of tether 20. In some embodiments, the worm gear configuration of tether reel 260 may comprise non-throated worm gears, enveloping worm gears and/or double enveloping worm gears.

Drive 280 includes a gear surface 290 that is configured for engagement with a lock 300, similar to lock 100 described herein, to resist and/or prevent movement of drive 280, as described herein. Drive 280 includes a socket 294 configured for a mating engagement with a driving tool (not shown) to rotate drive 280 and reel 262 relative to receiver 250 to adjust the tension of tether 20. Lock 300 is movable between a locked configuration, such that movement of reel 262 is resisted and/or prevented and a non-locked configuration, such that drive 280 and reel 262 are rotatable relative to receiver 250 to adjust the tension of tether 20, similar to lock 100 described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
at least one tether including a first end connectable with a first fastener connected with spinal tissue and a second end connectable with a second fastener connected with spinal tissue, the at least one tether having a tension between the fasteners; and
a member defining an axis and including an element disposed transverse relative to the axis, the element being engageable with the at least one tether and
the member further including a drive engageable with the element to adjust the tension.

2. A spinal construct as recited in claim 1, wherein the member further includes a lock engageable with the drive.

3. A spinal construct as recited in claim 2, wherein the lock is movable between a first configuration such that the lock engages the drive to resist and/or prevent movement thereof and a second configuration such that the drive is movable relative to the member.

4. A spinal construct as recited in claim 3, wherein the lock is resiliently biased to the first configuration.

5. A spinal construct as recited in claim 1, wherein the member further comprises a ratchet engageable with the drive to selectively adjust the tension.

6. A spinal construct as recited in claim 1, wherein the drive includes a gear surface and the member further comprises a lock including a pawl that engages the gear surface to resist and/or prevent movement of the drive.

7. A spinal construct as recited in claim 1, wherein the drive includes a gear surface engageable with a gear surface of the element to adjust the tension.

8. A spinal construct as recited in claim 7, wherein the gear surfaces comprise a bevel gear.

9. A spinal construct as recited in claim 7, wherein the gear surfaces comprise a worm gear.

10. A spinal construct as recited in claim 1, wherein the element defines a first axis and the drive defines a second axis disposed perpendicular relative to the first axis.

11. A spinal construct as recited in claim 1, wherein the element comprises a reel defining a cavity configured for disposal of the at least one tether.

12. A spinal construct as recited in claim 1, wherein at least one of the element and the drive are mounted with the member via a bearing to facilitate relative rotation thereof.

13. A spinal construct as recited in claim 1, wherein the member comprises a receiver of the first fastener, the second fastener or a third fastener connected with spinal tissue.

14. A tether reel comprising:
a reel engageable with an intermediate portion of a tether having a tension between ends thereof, the reel including a first gear surface, and
a drive including a second gear surface engageable with the first gear surface to rotate the reel and selectively adjust the tension.

15. A spinal construct as recited in claim 14, further comprising a lock engageable with the drive to resist and/or prevent movement of the drive.

16. A spinal construct as recited in claim 14, wherein the gear surfaces comprise a bevel gear.

17. A spinal construct as recited in claim 14, wherein the gear surfaces comprise a worm gear.

18. A spinal construct as recited in claim 14, wherein the reel defines a first axis and the drive defines a second axis disposed perpendicular relative to the first axis.

19. A spinal construct as recited in claim 14, further comprising a receiver of a bone screw, the receiver including the reel and the drive.

20. A spinal system comprising:
a first fastener connected with spinal tissue;
a second fastener connected with spinal tissue;
at least one tether including a first end connectable with the first fastener, a second end connectable with the second fastener and an intermediate portion, the at least one tether having a tension between the fasteners; and
at least one receiver defining an axis and including a reel disposed transverse relative to the axis, the reel being engageable with the at least one tether and
the at least one receiver further including a drive engageable with the reel to adjust the tension.
